SEQUENCE LISTING (6 sequences)

SEQ ID NO: 1 - DNA, 18 bp, synthetic construct
  source 1..18: mol_type = other DNA
tccagtatgactccactc

SEQ ID NO: 2 - DNA, 18 bp, synthetic construct
  source 1..18: mol_type = other DNA
atttctcgtggttcacac

SEQ ID NO: 3 - DNA, 24 bp, synthetic construct
  source 1..24: mol_type = other DNA
aaccgggaggccagctaaagaaca

SEQ ID NO: 4 - DNA, 23 bp, synthetic construct
  source 1..23: mol_type = other DNA
tgggcctacagaacagacagtgc

SEQ ID NO: 5 - DNA, 20 bp, synthetic construct
  source 1..20: mol_type = other DNA
gagaacctggagaagcagct

SEQ ID NO: 6 - DNA, 18 bp, synthetic construct
  source 1..18: mol_type = other DNA
ccgcggttggtccagtag

(19) United States
(12) Patent Application Publication
(10) Pub. No.: US 2026/0355265 A1
(43) Pub. Date: Nov. 12, 2026

(54) Composition for Improving Muscular Functions, and Preventing or Alleviating Muscle Disease Comprising Lactiplantibacillus Plantarum MTY101

(71) Applicant: FERMENTEC CO., LTD., Cheongju-si (KR)

(72) Inventor: Heung Yong Lee, Cheongju-si (KR)

(21) Appl. No.: 19/671,800

(22) Filed: May 8, 2026

(30) Foreign Application Priority Data
May 9, 2025 (KR) 10-2025-0060705

(51) Int. Cl. A61K 35/747; A61P 21/00

(52) U.S. Cl. CPC A61K 35/747; A61P 21/00

(57) ABSTRACT

The present disclosure relates to a composition comprising Lactiplantibacillus plantarum MTY101, and the composition comprising Lactiplantibacillus plantarum MTY101 has an effect of improving muscle strength and enhancing muscular functions, and thus may be used as a composition for preventing or alleviating muscle diseases or a composition for enhancing muscular functions.

### CROSS-REFERENCE TO RELATED APPLICATION

This present application claims the benefit of priority to Korean Patent Application No. 10-2025-0060705, entitled “Composition for preventing or improving muscle disease comprising Lactiplantibacillus plantarum MTY101” filed on May 9, 2025, in the Korean Intellectual Property Office, the entire disclosure of which is incorporated herein by reference.

### REFERENCE TO AN ELECTRONIC SEQUENCE LISTING

The contents of the electronic sequence listing (KAIP-009_BPX250022US.xml; Size: 6,375 bytes; and Date of Creation: May 6, 2026) is herein incorporated by reference in its entirety.

### FIELD

The present disclosure relates to the use of Lactiplantibacillus plantarum MTY101 for the prevention, alleviation, or treatment of muscle weakness, muscle loss, or muscular atrophy; and the use of Lactiplantibacillus plantarum MTY101 for enhancing muscular functions.

### BACKGROUND

Muscle accounts for about 40 to 50% of a person's body weight, and when a person reaches about 50 years of age, a synthetic rate of muscle proteins in myocytes slows down compared to a degradation rate, causing muscles to begin degenerating rapidly to be exposed to muscle loss diseases. Sarcopenia, which is a muscle loss disease, is a disease with a gradual decrease in muscle mass and muscle strength of the body, and the main symptoms are muscle loss and weakness. The sarcopenia causes the behavioral amount to be significantly reduced, which not only harms mental health but also lowers life satisfaction, and suffers easy injuries even during simple daily activities to lead to serious injuries. The sarcopenia is a disease that primarily occurs in the elderly, but has recently occurred across all generations. Currently, in Korea, one person in six people is elderly population aged 65 or older, and the number of patients with sarcopenia is on the rise due to the aging. Recently, muscle loss in the elderly has been recognized as a disease rather than natural aging, and has already been assigned and managed with a disease code in the United States (2016), WHO (2017), and Japan (2018), and the disease code was also assigned in Korea in 2021. It is known that if the sarcopenia occurs, the risk of death increases five-fold due to metabolic syndrome, including obesity, diabetes, and heart disease, osteoporosis, fractures, and the like. The sarcopenia has been assigned with a disease code, but has no therapeutic agent.

### SUMMARY

Accordingly, the present inventors isolated Lactiplantibacillus plantarum MTY101, which promoted muscle differentiation and formation, while researching the prevention, alleviation, or treatment of muscle weakness, muscle loss, or muscular atrophy. In addition, the present inventors confirmed that the isolated strain inhibited muscle degradation and simultaneously promoted muscle synthesis and muscle strength, and then completed the present disclosure.

Therefore, an object of the present disclosure is to provide a pharmaceutical composition for preventing or treating diseases caused by muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof.

Another object of the present disclosure is to provide a composition for preventing or alleviating muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof.

Yet another object of the present disclosure is to provide a composition for enhancing muscle or strengthening muscle, including a Lactiplantibacillus plantarum MTY101 (accession number: KCTC15786BP) strain, a culture medium thereof, a concentrate thereof, a lysate thereof, a dried product thereof, or a combination thereof.

Still another object of the present disclosure is to provide a method for preventing or treating diseases caused by muscle weakness, muscle loss, or muscular atrophy, including administering, to a subject in need thereof, a Lactiplantibacillus plantarum MTY101 (accession number: KCTC15786BP) strain, a culture medium thereof, a concentrate thereof, a lysate thereof, a dried product thereof, or a combination thereof.

Still another object of the present disclosure is to provide the use of a Lactiplantibacillus plantarum MTY101 (accession number: KCTC15786BP) strain, a culture medium thereof, a concentrate thereof, a lysate thereof, a dried product thereof, or a combination thereof, for the preparation of a drug for preventing or treating diseases caused by muscle weakness, muscle loss, or muscular atrophy.

In order to achieve the object, an aspect of the present disclosure provides a pharmaceutical composition for preventing or treating diseases caused by muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof.

Further, another aspect of the present disclosure provides a food composition for preventing or alleviating muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof.

Further, another aspect of the present disclosure provides a health functional food composition for preventing or alleviating muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof.

Further, another aspect of the present disclosure provides a feed composition for preventing or alleviating muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof.

Further, yet another aspect of the present disclosure provides a composition for enhancing muscle, including a Lactiplantibacillus plantarum MTY101 (accession number: KCTC15786BP) strain, a culture medium thereof, a concentrate thereof, a lysate thereof, a dried product thereof, or a combination thereof.

Further, still another aspect of the present disclosure provides a composition for strengthening muscle, including a Lactiplantibacillus plantarum MTY101 (accession number: KCTC15786BP) strain, a culture medium thereof, a concentrate thereof, a lysate thereof, a dried product thereof, or a combination thereof.

Further, still another aspect of the present disclosure provides a method for preventing or treating diseases caused by muscle weakness, muscle loss, or muscular atrophy, including administering, to a subject in need thereof, a Lactiplantibacillus plantarum MTY101 (accession number: KCTC15786BP) strain, a culture medium thereof, a concentrate thereof, a lysate thereof, a dried product thereof, or a combination thereof.

Further, still another aspect of the present disclosure provides the use of a Lactiplantibacillus plantarum MTY101 (accession number: KCTC15786BP) strain, a culture medium thereof, a concentrate thereof, a lysate thereof, a dried product thereof, or a combination thereof for the preparation of a drug for preventing or treating diseases caused by muscle weakness, muscle loss, or muscular atrophy.

According the present disclosure, a Lactiplantibacillus plantarum MTY101 strain has effects of preventing muscle weakness, muscle loss, or muscular atrophy and strengthening muscular functions. According the present disclosure, the Lactiplantibacillus plantarum MTY101 strain can be used variously in the field for treatment of muscle weakness, muscle loss, or muscular atrophy, and the field for strengthening of muscular functions.

### BRIEF DESCRIPTION OF THE DRAWINGS

FIG. 1 is a diagram illustrating results of analyzing a phylogenetic tree (left) and ANI (right) of Lactiplantibacillus plantarum MTY101 lactobacillus (marked with a box).

FIG. 2 is a diagram illustrating results of analyzing a YY1-promoter activity inhibitory function of a Lactiplantibacillus plantarum MTY101 lactobacillus culture medium by using muscle differentiation evaluation HTS (High-throughput screening); and PHF20-pYY1-Gluc assay in C2C12 myoblasts.

FIGS. 3A-3B are diagrams illustrating results of analyzing expression of muscle degradation (Atrogin-1, MuRF-1) and muscle synthesis (AKT phosphorylation: P-AKT) biomarkers at protein (3A) and mRNA (3B) levels, after treating a Lactiplantibacillus plantarum MTY101 lactobacillus culture medium for 24 hours in a dexamethasone-induced muscular atrophy C2C12-cell model.

FIG. 4A is a diagram illustrating results of analyzing cell viability, after treating C2C12 myoblasts with a Lactiplantibacillus plantarum MTY101 lactobacillus culture medium for 24 hours.

FIG. 4B is a diagram illustrating results of analyzing cell viability, after treating a Lactiplantibacillus plantarum MTY101 lactobacillus culture medium and dexamethasone together for 24 hours in a dexamethasone-induced muscular atrophy C2C12-cell model.

FIG. 5 is a diagram illustrating results of analyzing myotube formation through immunofluorescence staining of Myosin heavy chain (MyHC), after differentiating C2C12 myoblasts for 3 days in a muscle differentiation medium added with a Lactiplantibacillus plantarum MTY101 lactobacillus culture medium.

FIGS. 6A-6B are diagrams illustrating results of measuring body weight (6A) and grip strength (6B) during an administration period of Lactiplantibacillus plantarum MTY101 lactobacillus (probiotics) in a dexamethasone-induced muscular atrophy mouse model.

FIGS. 7A-7E are diagrams illustrating results of measuring total muscle mass (7A) and right hindlimb muscle mass (7B) after ending of administration of Lactiplantibacillus plantarum MTY101 lactobacillus (probiotics) in dexamethasone-induced muscular atrophy mice, and right hindlimb muscle mass (7C) before and after administration, a tissue staining result (7D) of the right hindlimb tibialis muscle, and a result of analyzing a cross section area thereof (7E).

### DETAILED DESCRIPTION

Hereinafter, the present disclosure will be described in detail.

According to another aspect of the present disclosure, the present disclosure provides a composition for preventing or alleviating muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof. The composition for preventing or alleviating muscle weakness, muscle loss, or muscular atrophy may be a food composition, a health functional food composition, or a feed composition.

The present inventors selected a strain having the activity of promoting muscle differentiation and formation from a lactobacillus culture medium library using a muscle differentiation evaluation HTS system in the same manner as Example 1 to be described below. The selected strain, Lactiplantibacillus plantarum MTY101 was deposited to the Biological Resources Center (Jeongeup-si, Jeollabuk-do, Korea) on Mar. 19, 2025, in accordance with the Treaty of Budapest, and assigned with accession number KCTC15786BP.

It was confirmed through the experiment that the selected Lactiplantibacillus plantarum MTY101 not only had no cytotoxicity, but also inhibited muscle degradation, promoted muscle synthesis, and improved muscle strength at the same time. Accordingly, Lactiplantibacillus plantarum MTY101 of the present disclosure may be used variously in the treatment field of muscle weakness diseases; fields related to increased muscle strength; the treatment field of muscle loss diseases; fields related to muscle gain; and fields related to strengthening muscular functions.

In a specific embodiment of the present disclosure, the strain preferably has one or more activities selected from the group consisting of promotion of myocyte differentiation, inhibition of myocyte death, improvement of muscle strength, improvement of exercise performance, and an increase in an internal mineral content, but the scope of the present disclosure is not limited thereto.

In the present disclosure, the culture medium refers to a medium containing a strain, by-products generated through a metabolism after the strain is cultured in a medium and ingests nutrients, and the like, and may specifically refer to a culture medium or culture of the strain. In addition, a concentrate or diluent of the culture medium, a dried product or freeze-dried product obtained by drying the culture medium, a modified product or purified product of the culture medium, a lysate obtained by crushing the culture medium, or a mixture thereof may also be included as an active ingredient in the composition of the present disclosure.

In a specific embodiment of the present disclosure, the strain may be included in the form of a live microbial cell, a dead microbial cell, or a combination thereof.

In the present disclosure, the microbial cell refers to the strain of the present disclosure itself and includes a strain itself or a strain isolated from the culture medium by culturing the strain, and may be a live microbial cell or a dead microbial cell. The microbial cells may be obtained by centrifuging the culture medium and taking a sunken portion in a lower layer, or obtained by sinking the culture medium to the lower layer due to gravity, still leaving the culture medium for a predetermined time, and then removing an upper liquid.

In a specific embodiment of the present disclosure, the prevention or alleviation of muscle weakness, muscle loss, or muscular atrophy may be muscle strengthening, improvement of muscle strength, muscle enhancement, promotion of muscle differentiation, or muscle regeneration.

In the present disclosure, the muscle differentiation refers to a process in which mononuclear myoblasts form multinuclear myotubes through fusion, and refers to inducing such a process. Cells in the differentiation stage that form myotubes are identified through the expression of markers such as Pax7, MyoD, and MyoG. By promoting the differentiation of myoblasts, myocytes increase, which may ultimately lead to an increase in muscle mass in a subject aspect.

In the present disclosure, the muscle regeneration refers to the ability of damaged muscle to recover to a normal condition. The muscle regeneration may be an effect caused by the promotion of myocyte differentiation to increase the absolute amount of myocytes, or increase the diameter of individual myotubes.

The food according to the present disclosure includes, for example, various kinds of foods, beverages, gum, tea, vitamin complexes, functional foods, and the like. In addition, the food includes special nutritional food (e.g., milk formulas, infant and baby foods, etc.), processed meat products, fish products, bean curd, jellied food, noodles (e.g., ramen, noodles, etc.), bread, health supplements, seasoned foods (e.g., soy sauce, soybean paste, red pepper paste, mixed soy sauce, etc.), sauces, confectionery (e.g., snacks), candies, chocolates, gums, ice creams, dairy products (e.g., fermented milk, cheese, etc.), other processed foods, kimchi, pickled foods (various types of kimchi, pickles, etc.), drinks (e.g., fruit drinks, vegetable drinks, soybean milk products, fermented drinks, etc.), natural seasonings (e.g., ramen soup, etc.), food additives, and the like, but is not limited thereto. The foods, drinks or food additives may be prepared by general preparation methods.

In the present disclosure, the health functional food refers to a group of foods to which added values are imparted by using physical, biochemical, or biotechnological methods to act and express the functions of the corresponding food for a specific purpose, or foods designed and processed to sufficiently express in the body in vivo regulatory functions regarding the regulation of biological defense rhythms, disease prevention, and recovery of the food composition. For the purpose of the present disclosure, the health functional food means foods for promoting muscle strengthening, muscle enhancement, muscle differentiation, or muscle regeneration activity.

When the composition of the present disclosure is used as a food or health functional food additive, the composition may be added as it is or used with other ingredients, and may be appropriately used according to a general method. The mixing amount of the active ingredients may be appropriately determined depending on the purpose of use. In general, when preparing foods or drinks, the composition of the present disclosure may be added in an amount of preferably 50 parts by weight or less, more preferably 25 parts by weight or less, based on the raw material. However, in the case of long-term intake for the purpose of health control and hygiene, the amount may be less than or equal to the above range, and has no problem in terms of safety, so that the active ingredient may be used even in an amount above the range.

The food or health functional food composition of the present disclosure may contain food-grade carriers, various common flavoring agents, natural carbohydrates, or the like as additional ingredients. Examples of the above-described natural carbohydrates may include general sugars, such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, erythritol, and the like. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.).

In the present disclosure, the feed refers to any natural or artificial constitutive diet, one meal, or the like eaten by animals or ingredients of the one meal, and the feed composition according to the present disclosure may be prepared in various types of feeds known in the art, and preferably may include concentrated feed, roughage and/or special feed, but is not limited thereto.

The feed composition of the present disclosure may be a feed additive composition. The feed additive composition includes substances added to the feed for the purpose of various effects, such as alleviation of disease symptoms in animals, nutrient supplementation and prevention of weight loss, improvement of fiber digestibility in feed, improvement of milk quality, prevention of reproductive disorders and improvement of conception rates, prevention of high-temperature stress during the summer, and the like.

The feed additive composition of the present disclosure correspond to supplementary feeds under the Feed Management Act, and may further include mineral preparations such as sodium bicarbonate, bentonite, magnesium oxide, and complex minerals; mineral preparations, which are trace minerals such as zinc, copper, cobalt, and selenium; vitamin preparations such as carotene, vitamins A, D, E, nicotinic acid, vitamin B complex; protected amino acid preparations such as methionine and lysine; protected fatty acid preparations such as fatty acid calcium salts; probiotics such as probiotics (lactobacillus), yeast cultures, and mold fermented products; yeast agents, and the like.

The concentrated feed among the feeds includes seed fruits including grains such as wheat, oats and corn; bran including rice bran, wheat bran, barley bran, etc. as by-products obtained by refining grains; residues such as seed cakes, which are by-products obtained from oil extraction of beans, rapeseed, sesame, linseed, and coco palms, and residual starches as a main ingredient of starch residues, which are the remainder after removing starch from sweet potatoes and potatoes; fish soluble obtained by concentrating fish meal, fish residues, and fresh liquids obtained from fish; animal feeds such as meat meal, blood meal, feather meal, skim milk powder, dried whey, which is obtained by drying whey as the balance when preparing cheese from milk and casein from skim milk, yeast, chlorella, and seaweed, but is not limited thereto.

The roughage among the feeds includes raw grass feed such as wild grass, grass, and green cutting; root vegetables such as turnips for feed, beets for feed, and Lutherbearer as a type of turnip; silage, which is stored feed made by filling a silo with green grass, green crops, and grains, and fermenting the silo with lactic acid; dried hay made by cutting and drying wild grass and grass; straw of breeding crops; and leaves of leguminous plants, but is not limited thereto. The special feed includes mineral feed such as oyster shells and rock salt; urea feed such as urea or its derivative, diureideisobutane; feed additives which are substances supplemented with ingredients that tend to be insufficient when only natural feed raw materials are mixed, or added in a small amount to mixed feed to improve the storage of feed; and dietary supplements, but is not limited thereto.

The feed additive composition according to the present disclosure may be prepared by adding one or more selected from the group consisting of Guanosine 5′-diphosphate 3′-diphosphate (ppGpp) deficient variant enteropathogenic E. coli (EPEC), a lysate thereof, and the strain culture, in an appropriate effective concentration range according to various feed preparation methods known in the art.

The feed additives according to the present disclosure may be applied without limitation to any subject for the purpose of the prevention or improvement of muscle weakness, muscle loss, or muscular atrophy. For example, the feed additives of the present disclosure may be applied even to any subject such as non-human animals such as monkeys, dogs, cats, rabbits, guinea pigs, rats, mice, cows, sheep, pigs, goats, etc., birds, fish, etc.

According to an aspect of the present disclosure, the present disclosure provides a pharmaceutical composition for preventing or treating diseases caused by muscle weakness, muscle loss, or muscular atrophy, including a Lactiplantibacillus plantarum MTY101 (accession number: KCTC15786BP) strain, a culture medium thereof, a concentrate thereof, a lysate thereof, a dried product thereof, or a combination thereof.

In a specific embodiment of the present disclosure, the diseases caused by muscle weakness, muscle loss, or muscular atrophy are preferably one or more selected from the group consisting of sarcopenia, atony, muscular atrophy, muscular dystrophy, muscle degeneration, myotonic dystrophy, amyotrophic lateral sclerosis, myasthenia, and cachexia, and may be applied without limitation to any symptoms, diseases, or disorders caused by muscle weakness, muscle loss, or muscular atrophy.

In the present disclosure, the prevention refers to any action of suppressing or delaying the onset of diseases caused by muscle weakness, muscle loss, or muscular atrophy by the administration of the pharmaceutical composition according to the present disclosure. In addition, in the present disclosure, the treatment refers to any action that improves or beneficially alters the symptoms of diseases caused by muscle weakness, muscle loss, or muscular atrophy by the administration of the pharmaceutical composition according to the present disclosure.

The pharmaceutical composition of the present disclosure may further include a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, and the like. The pharmaceutically acceptable additive according to the present disclosure is preferably included in an amount of 0.1 to 90 parts by weight based on the composition, but is not limited thereto.

In addition, the pharmaceutical composition of the present disclosure may be administered in various oral or parenteral formulations during actual clinical administration. In the case of the formulation, the pharmaceutical composition may be prepared using diluents or excipients such as commonly used fillers, extenders, binders, wetting agents, disintegrants, and surfactants, and it is preferable to use suitable formulations known in the art. The carriers, the excipients, and the diluents which may be included in the composition include lactose, dextrose, sucrose, oligosaccharide, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, mineral oil, etc.

Solid formulations for oral administration include tablets, pills, powders, granules, capsules, and the like, and these solid formulations are prepared by mixing at least one excipient, for example, starch, calcium carbonate, sucrose or lactose, gelatin, and the like. Further, lubricants such as magnesium stearate and talc may be used in addition to simple excipients. In addition, liquid formulations for oral administration may correspond to a suspension, an oral liquid, an emulsion, a syrup, and the like, and may include various excipients, for example, a wetting agent, a sweetener, an aromatic agent, a preserving agent, and the like, in addition to water and liquid paraffin which are commonly used as simple diluents.

Formulations for parenteral administration include sterile aqueous solutions, non-aqueous solvents, suspensions, emulsions, lyophilized preparations, and suppositories. As the non-aqueous solution and the suspension, propylene glycol, polyethylene glycol, vegetable oil such as olive oil, injectable ester such as ethyl oleate, and the like may be used. As a base of the suppository, witepsol, macrogol, tween 61, cacao butter, laurinum, glycerogelatin, and the like may be used. The parenteral administration may be performed using external skin application or intraperitoneal injection, rectal injection, subcutaneous injection, intravenous injection, intramuscular injection, or thoracic injection methods.

The dose range of the pharmaceutical composition of the present disclosure varies depending on the patient's weight, age, sex, health condition, diet, time of administration, method of administration, excretion rate, and severity of a disease, and may be administered once a day or divided and administered several times.

The pharmaceutical composition of the present disclosure may be administered to a subject through various routes.

The pharmaceutical composition of the present disclosure may be used alone or in combination with surgery, radiation therapy, hormone therapy, chemotherapy, and methods of using biological response modifiers, for the prevention or treatment of diseases caused by muscle weakness, muscle loss, or muscular atrophy.

According to yet another aspect of the present disclosure, the present disclosure provides a composition for enhancing muscle or strengthening muscle, including a Lactiplantibacillus plantarum MTY101 (accession number: KCTC15786BP) strain, a culture medium thereof, a concentrate thereof, a lysate thereof, a dried product thereof, or a combination thereof.

In the present disclosure, the muscle enhancement and the muscle strengthening are preferably caused by promotion of myocyte differentiation, inhibition of myocyte death, improvement of muscle strength, improvement of exercise performance, or an increase in internal mineral content.

In an embodiment of the present disclosure, the strain according to the present disclosure was orally administered to a mouse. As a result, the muscle and internal mineral content of the mouse were significantly increased, and the hip-shin muscle mass increased by up to 29% compared to before administration. In addition, in another embodiment of the present disclosure, as a result of oral administration of the strain according to the present disclosure to aged mice, a significant increase in grip strength and treadmill performance was confirmed. The result indicates that the strain according to the present disclosure positively alters changes in body composition and improves exercise ability, and thus may be used as a composition for muscle enhancement or muscle strengthening.

The composition for muscle enhancement or muscle strengthening of the present disclosure may be prepared in various forms, such as a food composition, a health functional food composition, and a food additive composition, and may be used in the form of adjuvants and the like for muscle formation and muscle strengthening in the general public as well as for muscle loss caused by aging or disease.

According to still another aspect of the present disclosure, the present disclosure provides a method for preventing or treating diseases caused by muscle weakness, muscle loss, or muscular atrophy, including administering, to a subject in need thereof, a Lactiplantibacillus plantarum MTY101 (accession number: KCTC15786BP) strain, a culture medium thereof, a concentrate thereof, a lysate thereof, a dried product thereof, or a combination thereof.

The Lactiplantibacillus plantarum MTY101 (accession number; KCTC15786BP) strain has one or more activities selected from the group consisting of promotion of myocyte differentiation, inhibition of myocyte death, improvement of muscle strength, improvement of exercise performance, or an increase in internal mineral content.

In a specific embodiment of the present disclosure, the prevention or treatment of the diseases caused by muscle strength and muscle loss; or muscular atrophy may be muscle strengthening, improvement of muscle strength, muscle enhancement, promotion of muscle differentiation, or muscle regeneration.

In a specific embodiment of the present disclosure, the diseases caused by muscle strength and muscle loss; or muscular atrophy are preferably one or more selected from the group consisting of sarcopenia, atony, muscular atrophy, muscular dystrophy, muscle degeneration, myotonic dystrophy, amyotrophic lateral sclerosis, myasthenia, and cachexia, and may be applied without limitation to any symptoms, diseases, or disorders caused by muscle weakness, muscle loss, or muscular atrophy.

The Lactiplantibacillus plantarum MTY101 (accession number; KCTC15786BP) strain, the culture medium thereof, the concentrate thereof, the lysate thereof, the dried product thereof, or the combination thereof may be administered to a subject in need thereof in a therapeutically effective amount. The subject is preferably mammals including humans, and may include all patients who have received prevention or treatment for diseases caused by muscle weakness, muscle loss, or muscular atrophy, and patients who need to receive prevention or treatment for diseases caused by muscle weakness, muscle loss, or muscular atrophy; and may also include patients who have undergone other therapies or treatments for the prevention or treatment of diseases caused by muscle weakness, muscle loss, or muscular atrophy.

In addition, the present disclosure may be used and treated in combination with existing drugs or treatment methods for the prevention or treatment of diseases caused by muscle weakness, muscle loss, or muscular atrophy. The combined treatment with the present disclosure may be used simultaneously or sequentially with other drugs or treatment methods for the prevention or treatment of diseases caused by muscle weakness, muscle loss, or muscular atrophy.

According to still another aspect of the present disclosure, the present disclosure provides a method for strengthening muscle and alleviating muscular atrophy, the method comprising administering, to a subject in need thereof, a composition comprising a Lactiplantibacillus plantarum MTY101 (accession number: KCTC15786BP) strain, a culture medium thereof, a concentrate thereof, a lysate thereof, a dried product thereof, or a combination thereof.

The composition may be a food composition, a health functional food composition, or a feed composition.

The Lactiplantibacillus plantarum MTY101 (accession number: KCTC15786BP) strain has one or more activities selected from the group consisting of promotion of myocyte differentiation, inhibition of myocyte death, improvement of muscle strength, improvement of exercise performance, or an increase in internal mineral content.

In a specific embodiment of the present disclosure, the alleviation of the muscular atrophy may be muscle enhancement, promotion of muscle differentiation, or muscle regeneration.

According to still another aspect of the present disclosure, the present disclosure provides the use of a Lactiplantibacillus plantarum MTY101 (accession number: KCTC15786BP) strain, a culture medium thereof, a concentrate thereof, a lysate thereof, a dried product thereof, or a combination thereof for the preparation of a drug for preventing or treating diseases caused by muscle weakness, muscle loss, or muscular atrophy.

Duplicated contents will be omitted in consideration of the complexity of the present specification, and terms not defined otherwise herein have the meanings commonly used in the art to which the present disclosure pertains.

Hereinafter, the present disclosure will be described in more detail through Examples. These Examples are just illustrative of the present disclosure, and it will be apparent to those skilled in the art that the scope of the present disclosure is not to be construed as being limited by these Examples.

### Example 1. Selection and Characterization of Lactobacillus Strain Promoting Muscle Differentiation and Formation

Lactiplantibacillus plantarum MTY101 having activity of promoting muscle differentiation and formation was selected from a lactobacillus culture medium library. The Lactiplantibacillus plantarum MTY101 was deposited from the present inventors to the Biological Resources Center (Jeongeup-si, Jeollabuk-do, Korea) on Mar. 19, 2025, in accordance with the Treaty of Budapest, and assigned with accession number KCTC15786BP.

### 1-1. Genome-Based Analysis of MTY101 Strain

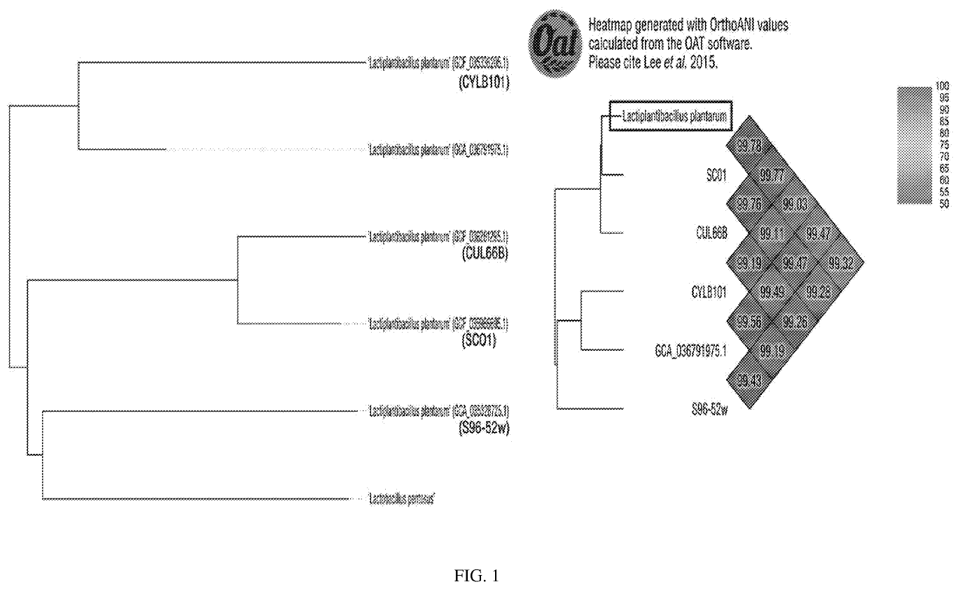

Based on the whole genome of the MTY101 strain isolated from kimchi, a phylogenetic tree and ANI were analyzed and shown in FIG. 1. It was confirmed that the MTY101 strain of the present disclosure had a genome of 3.34 Gb analyzed into 47 contigs and contained 3,173 genes. This gene contained 3,101 CDS, 63 tRNAs, 8 rRNAs, and 1 tmRNA.

### 1-2. Antibiotic Susceptibility of MTY101 Strain

The antibiotic susceptibility of Lactiplantibacillus plantarum MTY101 was evaluated using a minimum inhibitory concentration (MIC) test strip (KisanBio, Korea). The antibiotic susceptibility of the strain to eight antimicrobial agents, including ampicillin, vancomycin, gentamicin, kanamycin, streptomycin, erythromycin, clindamycin, and tetracycline, was tested according to the European Food Safety Authority (EFSA) guidelines. Specifically, cells were aerobically cultured in an MRS medium at 37 °C for 18 hours, harvested by centrifugation at 11,000 rpm for 5 minutes, and washed three times with PBS (pH 7.4). The washed cells were resuspended in PBS in McFarland standard 0.5. The cell suspension was inoculated onto BHI agar containing a cotton swab. The plate was dried for 10 to 15 minutes, and the MIC test strip was placed on the agar surface. Next, the plate was incubated at 37 °C for 48 hours, and the MIC was read based on the intersection of the MIC scale on the test strip and the elliptical area of growth inhibition. Cutoff values for various antibiotics were evaluated according to EFSA guidelines. The results of evaluating antibiotic resistance using MIC and hemolysis tests were shown in Table 1.

As a result of antibiotic susceptibility testing, it was confirmed that the strain exhibited susceptibility to most antibiotics, but showed resistance to ampicillin (AM) and chloramphenicol (CL), and inherent resistance to vancomycin (VM). In particular, it was confirmed that bacterial growth was inhibited at a minimum inhibitory concentration (MIC) for erythromycin (EM) which was at least half lower than cut-off values.

TABLE 1

| | | Antimicrobial susceptibility (MIC) (μg/mL) | | | | | | | | |
|---|---|---|---|---|---|---|---|---|---|---|
| Strain name | Strain | AM | VM | GM | KM | SM | EM | CM | TC | CL |
| Cut-off values | | 2 | n.r. | 16 | 64 | n.r. | 1 | 2 | 32 | 8 |
| L. plantarum | MYT101 | 3 | > | 0.5 | 32 | 32 | 0.125 | 1.5 | 16 | 12 |

* n.r. not required; Ampicillin (AM); Vancomycin (VM); Gentamicin (GM); Kanamycin (KM); Streptomycin (SM); Erythromycin (EM); Clindamycin (CM); Tetracycline (TC); Chloramphenicol (CL)

### 1-3. Activity of Promoting Muscle Differentiation and Formation of MTY101 Strain

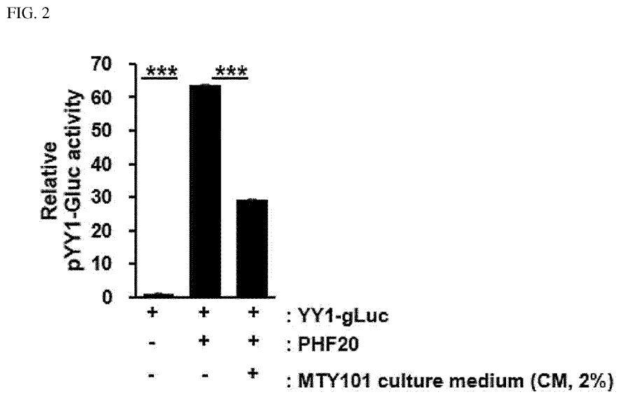

1×10⁴ C2C12 mouse myoblasts were mixed with a culture medium (DMEM, 10% FBS, 1% AA), seeded into a 96-well plate, and cultured in a cell incubator for 24 hours. After 24 hours, YY1-promoter-Gaussia luciferase DNA (pYY1-Gluc, 50 ng) and Flag-PHF20 DNA (200 ng) were introduced into cells using Lipofectamine 3000 (cat #L3000001, Invitrogen) according to the manufacturer's manual. After 24 hours of intracellular introduction, the culture medium was replaced with a fresh culture medium, and 1×10⁸ CFU of a Lactiplantibacillus plantarum MTY101 culture medium (CM) was treated at a concentration of 2% of the cell culture medium and additionally cultured for 24 hours. After the culture was completed, the activity of Gaussia luciferase was measured. The Gaussia luciferase activity was measured using a dedicated measurement reagent (cat #LF062, Genecopenia), and the experiment was performed according to the manufacturer's manual. In addition, control groups for the experiment were YY1-promoter-Gaussia luciferase; and YY1-promoter-Gaussia luciferase+PHF20, which were not treated with the culture medium. As comparative experimental groups, culture mediums of nine different Lactiplantibacillus plantarum strains distributed from the Korea Research Institute of Bioscience and Biotechnology were treated in the same manner, and then the YY1 promoter activities were compared. The result of measuring the YY1 promoter activities was shown in FIG. 2. The graph shows the mean and standard deviation of three repeated measurements of each sample, and a difference between samples was tested using a two-sample t-test (independent sample t-test) in the Excel statistics (***p<0.001).

As shown in FIG. 2, the MTY101 lactobacillus culture medium significantly inhibited YY1 transcriptional activation induced by PHF20 (P<0.001). In addition, it was confirmed that the MTY101 strain exhibited very excellent YY1 promoter inhibitory activity compared to other Lactiplantibacillus plantarum strains, whereas even in the same Lactiplantibacillus plantarum, the nine Lactiplantibacillus plantarum strains used as the comparative experimental groups showed no YY1 promoter inhibitory activity at all.

Accordingly, the Lactiplantibacillus plantarum MTY101 strain of the present disclosure was selected as a strain having excellent activity of promoting muscle differentiation and formation, and the strain of the present disclosure and a culture medium thereof were used in additional experiments below.

### Example 2. Confirmation of Biomarkers Related to Promotion of Muscle Differentiation and Inhibition of Muscle Degradation According to Treatment with Strain Culture Medium

### 2-1. Dexamethasone-Induced Muscular Atrophy C2C12-Cell Model

C2C12 myoblasts were seeded into a 6-well plate and cultured in a culture medium until the plate was filled to at least 90%, and then the medium was replaced with a myocyte differentiation medium (DMEM, 2% Horse Serum, 1% AA) to induce myotube differentiation. The differentiation was performed for 5 days, and the medium was replaced with a fresh differentiation medium every day. To induce muscular atrophy, 100 μM dexamethasone (DEX) was treated at day 4, and at this time, 1×10⁸ CFU of the MTY101 lactobacillus culture medium was simultaneously treated according to the concentrations shown in each diagram. After 24 hours of dexamethasone treatment, the medium was removed and the cells were harvested after washing once with PBS.

### 2-2. Protein Expression Analysis

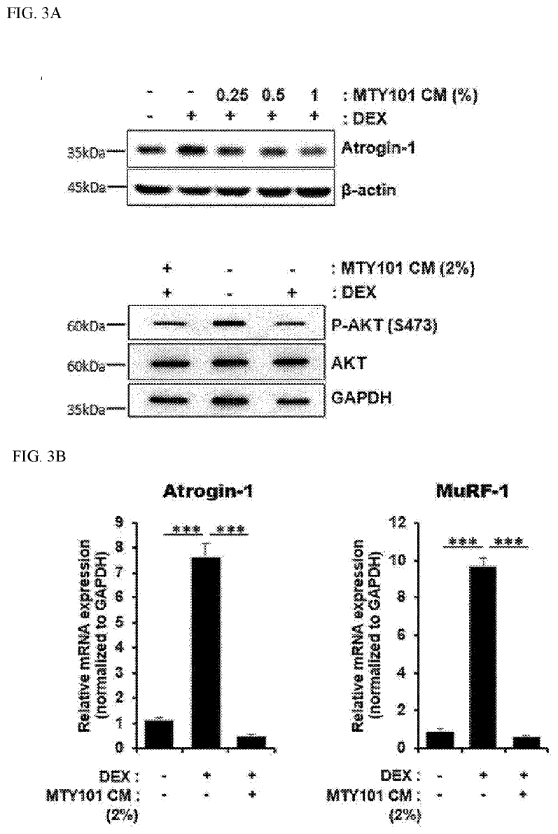

The intracellular protein extraction was performed by extracting proteins according to the manufacturer's protocol using a protein extraction solution (PRO-PREP™ Protein Extraction Solution, iNtRON), and then the proteins were isolated using 12 to 15% SDS-PAGE. Thereafter, the isolated proteins were transferred to a PVDF membrane, membrane blocking was performed with 5% skim milk/TBS-T (0.05% Tween-20) at room temperature for 1 hour, and then Western blotting was performed using the following antibodies: Atrogin-1(#ab92281) of Abcam; p-AKT(#4060), AKT(#9272) of Cell Signaling Technology; GAPDH(sc-25778) of Santa Cruz; β-actin(#A2228) of Sigma Aldrich. GAPDH and β-actin were used as an internal control to correct total protein amounts between samples. The results of the protein expression analysis were illustrated in FIGS. 3A-3B.

As shown in FIG. 3A, it was confirmed that the expression of the muscle degradation biomarker Atrogin-1, which was increased by dexamethasone treatment, was decreased depending on a treatment concentration of the MTY101 lactobacillus culture medium. Conversely, it was confirmed that the phosphorylation of the muscle synthesis biomarker AKT, which was decreased by dexamethasone treatment, was inhibited by treatment with the MTY101 lactobacillus culture medium.

2-3. mRNA Expression Analysis

RNA isolation, cDNA synthesis, and Q-PCR samples were performed using the AccuPrep® Universal RNA Extraction Kit, AccuPower® RT PreMix, and AccuPower® 2X GreenStar™ qPCR MasterMix (Bioneer, Korea), respectively, according to the protocol of each manufacturer. The PCR reaction was performed using an AriaMx real-time PCR instrument (Agilent) under the following conditions: 1) initial denaturation step, 3 min at 95 °C, 2) denaturation step, 15 sec at 95 °C, 3) annealing step, 30 sec at 60 °C, 4) extension step, 30 sec at 72 °C, and steps 2) to 4) performed for 40 cycles.

The primer sequences used for PCR were as follows: mouse GAPDH, Forward: 5′-TCCAGTATGACTCCACTC-3′ (SEQ ID NO: 1), Reverse: 5′-ATTTCTCGTGGTTCACAC-3′ (SEQ ID NO:2); mouse Atrogin-1, Forward: 5′-AACCGGGAGGCCAGCTAAAGAACA′ (SEQ ID NO: 3), Reverse: 5′-TGGGCCTACAGAACAGACAGTGC-3′ (SEQ ID NO: 4); mouse MuRF-1: Forward: 5′-GAGAACCTGGAGAAGCAGCT′ (SEQ ID NO: 5), Reverse: 5′-CCGCGGTTGGTCCAGTAG-3′ (SEQ ID NO: 6).

The mRNA expression of each gene was calculated using a 2^{-ΔΔCt} method after being corrected with the expression level of a control gene GAPDH, and shown in a graph of the mean and standard deviation (n=3/sample).

As shown in FIG. 3B, it was confirmed that the mRNA expression of the muscle degradation biomarkers Atrogin-1 and MuRF-1 was increased by dexamethasone treatment (***p<0.001). The increase induced by dexamethasone was inhibited by treatment with the MTY101 lactobacillus culture medium (***p<0.001).

### Example 3. Confirmation of Cytotoxicity and Apoptosis Inhibitory Effects of Strain Culture Medium

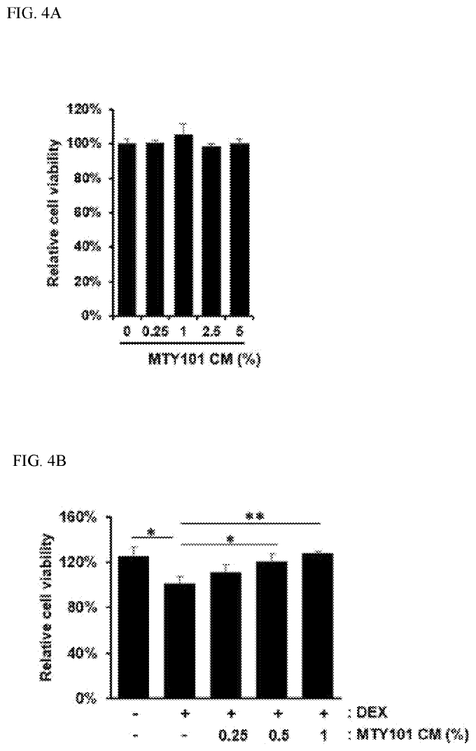

1×10⁴ C2C12 cells were seeded into a 96-well plate and cultured in a culture medium for 24 hours. After 24 hours, the medium was replaced with a fresh medium, and 1×10⁸ CFU of the MTY101 lactobacillus culture medium was treated at a concentration of 0.25 to 5% and cultured for 24 hours. After 24 hours, cell viability was measured using an MTT assay (Ez-Cytox, cat #EZ-3000, DoGenBio). The results were shown in FIG. 4A (mean±standard deviation, n=3/sample).

As shown in FIG. 4A, it was confirmed that the MTY101 lactobacillus culture medium showed cell viability similar to the control group at all treatment concentration to have no cytotoxicity.

To confirm whether the MTY101 lactobacillus culture medium inhibited dexamethasone-induced apoptosis in myotubes, myotubes differentiated for 5 days were treated with 1×10⁸ CFU of the MTY101 lactobacillus culture medium at a concentration of 0.25 to 1% and simultaneously treated with dexamethasone, and then cultured in a differentiation medium for 24 hours. The apoptosis was measured using MTT assay, and the results were shown in FIG. 4B (mean±standard deviation, n=3/sample).

As shown in FIG. 4B, a decrease in cell viability induced by dexamethasone (*p<0.05) was inhibited depending on the treatment concentration of the MTY101 lactobacillus culture medium (*p<0.05, **p<0.01).

### Example 4. Effect of Promoting Myotube Formation of MTY101 Lactobacillus Culture Medium

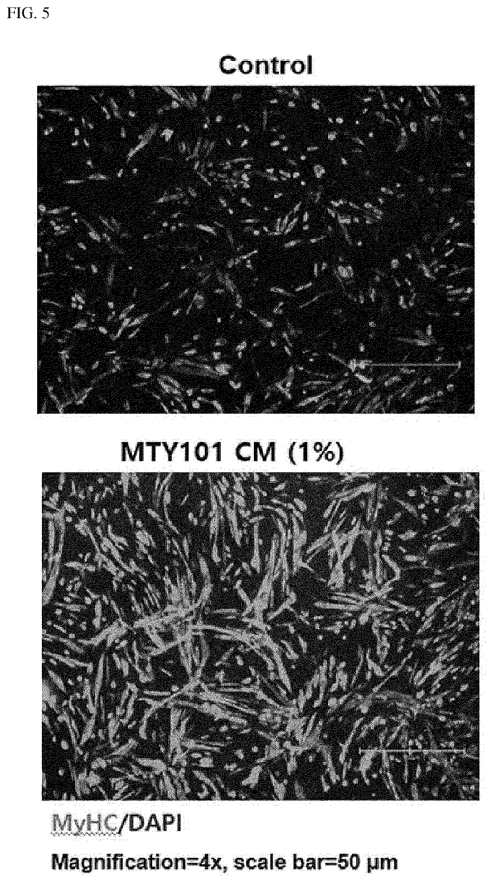

In Example 4, a myotube formation promoting effect of the MTY101 lactobacillus culture medium was confirmed. Specifically, a sterile glass coverslip was placed in a 24-well plate, and 5×10⁴ C2C12 cells were seeded per well and cultured in the culture medium for 24 hours. After 24 hours, the culture medium was replaced with a differentiation medium, and the cells were differentiated for 3 days. During the differentiation period, 1×10⁸ CFU of the MTY101 lactobacillus culture medium was contained in the differentiation medium at a concentration of 1%, and the differentiation medium was replaced daily. After the differentiation was completed, the cells were washed twice with PBS and fixed in 4% paraformaldehyde at room temperature for 15 minutes. Then, the cells were washed three times with PBS, reacted with 0.2% Triton x-100/PBS for 10 minutes at room temperature, washed three times with PBS, and blocked with 1% BSA/PBS for 1 hour at room temperature. Thereafter, an anti-MYH antibody (Santacruz, sc-376157) was diluted 1:1000 in 1% BSA and reacted with the cells at 4 °C for 24 hours, washed three times with PBS, and then reacted with an Alexa Fluor™ Plus 488-secondary antibody (Invitrogen, Cat #A32723: 1:5000, diluted with PBS) for 1 hour at room temperature under light-blocked conditions. Fluorescently stained myotubes were imaged using a Zeiss fluorescence microscope after staining the cell nuclei with DAPI VECTASHIELD (St. Louis, USA) and mounting the coverslip on a glass slide. The fluorescent staining result was shown in FIG. 5.

As shown in FIG. 5, it was confirmed that myotube formation was promoted in the group treated with the MTY101 lactobacillus culture medium compared to an untreated group.

### Example 5. Effect of Improving Grip Strength of MTY101 Lactobacillus (Probiotics) in Dexamethasone-Induced Muscular Atrophy Mouse Model

### 5-1. Dexamethasone-Induced Muscular Atrophy Mouse Model

7-week-old male C57BL/6N mice were adapted in a rearing room for 1 week, and then the mice were randomly divided into a Control group, a DEX group, and an MTY101 group (N=5/group), and the DEX group and the MTY101 group were intraperitoneally injected with dexamethasone (25 mg/kg) daily for 9 days to induce muscular atrophy. Thereafter, the Control group and the DEX group were orally administered with PBS and the MTY101 group was orally administered with 1×10⁸ CFU of the MTY101 lactobacillus (probiotics) suspended in 100 μl PBS every day for 14 days. The grip strength was measured on days 12 and 23 after dexamethasone administration, and the body weight was measured periodically (on days 0, 2, 4, 6, 11, 19, and 24). The body composition was analyzed using dual energy x-ray absorptiometry (DXA) on day 11 (before administration of a test substance) and day 24 (after administration of the test substance) from the starting of the test. Then, on the next day, day 25, the mouse was euthanized and the tibialis anterior (TA) tissue of the hindlimb was isolated.

### 5-2. Evaluation of Grip Strength

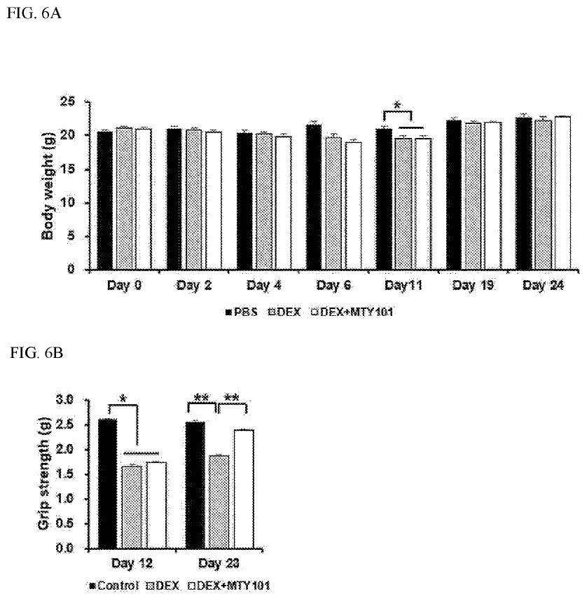

Results of evaluating changes in body weight and grip strength of mice during a test period were shown in FIGS. 6A and 6B, respectively (mean±standard error, n=5 mice/group).

As shown in FIG. 6A, on day 11 of dexamethasone administration, it was confirmed that the body weights of the dexamethasone administered groups (DEX group and MTY101 group) decreased compared to the Control group (*p<0.05). This weight loss indicates that a muscular atrophy model induced by dexamethasone has been constructed. Subsequently, this weight loss was restored to the level of the Control group.

As shown in FIG. 6B, on day 12, the next day after the end of dexamethasone administration, it was confirmed that the grip strength of the groups administered with dexamethasone (DEX group and MTY101 group) was significantly decreased compared to the Control group (*P<0.05). In addition, as a result of measuring the grip strength on day 23, it was confirmed that the grip strength of the MTY101 group was restored to the level of the Control group (**P<0.01), but the grip strength of the DEX group was significantly decreased compared to the Control group (**P<0.01). These results indicate that the decrease in grip strength by dexamethasone administration is inhibited by the administration of the MTY101 lactobacillus (probiotics).

### Example 6. Effect of Improving Muscle Mass of MTY101 Lactobacillus (Probiotics)

### 6-1. Increase in Muscle Mass According to Administration of MTY101 Lactobacillus (Probiotics)

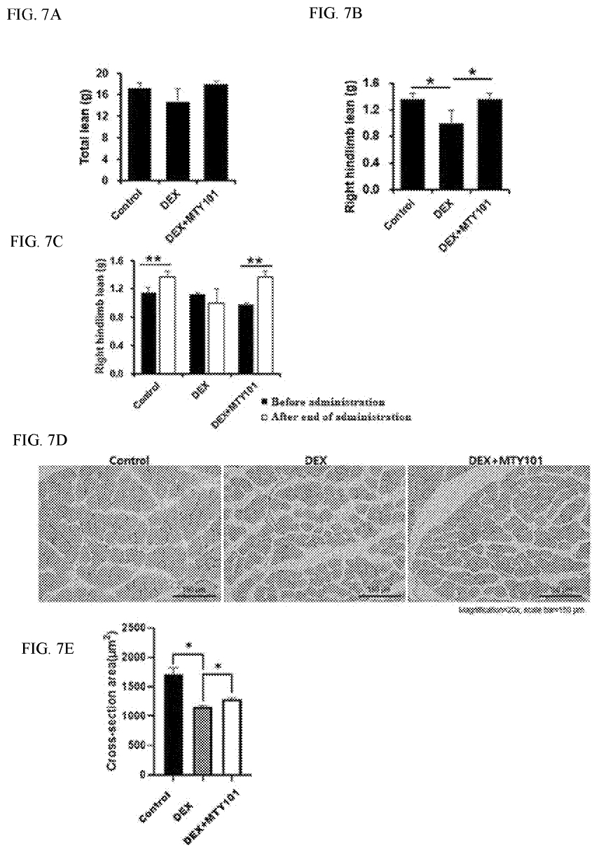

To confirm the effect of administration of MTY101 lactobacillus (probiotics) on an increase in muscle mass, changes in muscle mass before and after the administration of the test substance were confirmed through body composition analysis using DXA, after the administration of MTY101 lactobacillus (probiotics) was completed in the same manner as in Example 5. The results of measuring total muscle mass and right hindlimb muscle mass after end of the administration of Lactiplantibacillus plantarum MTY101 lactobacillus (probiotics), and right hindlimb muscle mass before and after administration in dexamethasone-induced muscular atrophy mice were shown in FIGS. 7A to 7C, respectively.

As shown in FIGS. 7A and 7B, the total muscle mass after end of the administration was found to have no significant different between the groups. On the other hand, when comparing the right hindlimb muscle mass, it was confirmed that the muscle mass of the DEX group was significantly decreased compared to the Control group (*P<0.05). In addition, a decrease in the hindlimb muscle mass by dexamethasone was inhibited by the MTY101 administration (*P<0.05).

As shown in FIG. 7C, changes in right hindlimb muscle mass before and after administration of the test substance to each group were shown. The Control group showed an increase in muscle mass with increasing week-age (**P<0.01), but the DEX group did not show an increase in muscle mass. On the other hand, the MTY101 group showed a significant increase in muscle mass after administration compared to before administration, despite the administration of dexamethasone (**P<0.01).

### 6-2. Increase in Muscle Cross-Section Area According to Administration of MTY101 Lactobacillus (Probiotics)

In the same manner as in Example 5, the hindlimb tibialis tissue of a dexamethasone-induced muscular atrophy mouse administered with MTY101 lactobacillus (probiotics) was fixed with 4% paraformaldehyde at 4 °C for 48 hours, and then embedded in paraffin to fabricate a paraffin block. The paraffin block was cut into 4 μm-thick sections and stained with hematoxylin and eosin (H&E). H&E images were captured using a microscope (EVOS™ M5000, Invitrogen), and the muscle cross-section area was analyzed using Image J software. The results of observing the stained sections were shown in FIG. 7D, and the graph drawn up based thereon was shown in FIG. 7E (mean±standard error, n=5/group).

As shown in FIGS. 7D and 7E, it was confirmed that the muscle cross-section area of the DEX group was significantly decreased compared to the Control group (*P<0.05). In addition, it was confirmed that the muscle cross-section area of the MTY101 group was significantly increased compared to the DEX group (*P<0.05). This indicates that the muscle loss caused by dexamethasone is inhibited by the administration of MTY101 lactobacillus (probiotics).

### Example 7. Statistical Analysis

Differences between samples were tested using a paired t-test or unpaired t-test of the Excel statistics, and shown as *; P-value (reliability)<0.05, **; p<0.01, ***; p<0.001. P<0.05 was interpreted as a statistically significant difference. If there was no statistically significant difference, it was represented as ns.

In summary, the present inventors selected a strain of the genus Lactobacillus that promoted muscle differentiation and formation. In addition, it was confirmed through the experiment that the selected strain of the genus Lactobacillus not only had no cytotoxicity, but also inhibited muscle degradation and simultaneously promoted muscle synthesis. Therefore, the strain of the genus Lactobacillus of the present disclosure may be used variously in the fields of treating muscle loss diseases and the fields related to muscle gain.

As described above, specific parts of the present disclosure have been described in detail, and it will be apparent to those skilled in the art that these specific techniques are merely preferred embodiments, and the scope of the present disclosure is not limited thereto. Therefore, the substantial scope of the present disclosure will be defined by the appended claims and their equivalents.

### Deposit of Biological Material

Lactiplantibacillus plantarum MTY101 of the present disclosure was deposited to the Biological Resources Center (Jeongeup-si, Jeollabuk-do, Korea) on Mar. 19, 2025, in accordance with the Treaty of Budapest, and assigned with accession number KCTC15786BP.

If the patent is granted, all restrictions on the public use of the deposited culture medium are irrevocably lifted, and the applicant retains the right to request so that a request for the deposit is made in accordance with 37 CFR § 1.808 (b).

The deposit will be kept for the longer period of 30 years from the deposit date, 5 years from the date of the last sample request, or the patent valid duration, and will be maintained with all possible attention required to keep it viable and uncontaminated in any case.

The applicant requests samples of the deposited biological material described in the present application to be provided only to independent experts in accordance with relevant patent laws, e.g., Rule 32 of the European Patent Convention (EPC), Rule 13 (1) and Articles 6 and 7 of Addenda 1 of the UK Patent Rules 2007 (Revised), and similar provisions applicable to other designated countries, for all designated and/or elected countries, and within the range legally permitted range under the laws of the countries.

1. A method for preventing or treating diseases caused by muscle strength and muscle loss; or muscular atrophy, the method comprising administering, to a subject in need thereof, a Lactiplantibacillus plantarum MTY101 (accession number: KCTC15786BP) strain, a culture medium thereof, a concentrate thereof, a lysate thereof, a dried product thereof, or a combination thereof.

2. The method of claim 1, wherein the strain has one or more activities selected from the group consisting of promotion of myocyte differentiation, inhibition of myocyte death, improvement of muscle strength, improvement of exercise performance, or an increase in internal mineral content.

3. The method of claim 1, wherein the prevention or treatment of the diseases caused by muscle strength and muscle loss; or muscular atrophy is muscle strengthening, improvement of muscle strength, muscle enhancement, promotion of muscle differentiation, or muscle regeneration.

4. The method of claim 1, wherein the diseases caused by muscle strength and muscle loss; or muscular atrophy are one or more selected from the group consisting of sarcopenia, atony, muscular atrophy, muscular dystrophy, muscle degeneration, myotonic dystrophy, amyotrophic lateral sclerosis, myasthenia, and cachexia.

5. A method for strengthening muscle and alleviating muscular atrophy, the method comprising administering, to a subject in need thereof, a composition comprising a Lactiplantibacillus plantarum MTY101 (accession number: KCTC15786BP) strain, a culture medium thereof, a concentrate thereof, a lysate thereof, a dried product thereof, or a combination thereof.

6. The method of claim 5, wherein the composition is a food, health functional food, or feed composition.

7. The method of claim 5, wherein the strain has one or more activities selected from the group consisting of promotion of myocyte differentiation, inhibition of myocyte death, improvement of muscle strength, improvement of exercise performance, or an increase in internal mineral content.

8. The method of claim 5, wherein the alleviation of the muscular atrophy is muscle enhancement, promotion of muscle differentiation, or muscle regeneration.
